(19) United States
(12) Patent Application Publication
(10) Pub. No.: US 2013/0236506 A1
(43) Pub. Date: Sep. 12, 2013

(54) AMNION AND CHORION CONSTRUCTS AND USES THEREOF IN OB-GYN SURGERY

(71) Applicant: AFCELL MEDICAL, Parsippany, NJ (US)

(72) Inventor: Robin R. YOUNG, Wayne, PA (US)

(73) Assignee: AFCELL MEDICAL, Parsippany, NJ (US)

(21) Appl. No.: 13/790,712

(22) Filed: Mar. 8, 2013

Related U.S. Application Data
(60) Provisional application No. 61/608,397, filed on Mar. 8, 2012.

(51) Int. Cl. A61L 27/36; A61L 27/54

(52) U.S. Cl. CPC A61L 27/3604; A61L 27/54
USPC 424/400; 424/582

(57) ABSTRACT

A construct for use in an OB/GYN surgery is described. The construct contains an allograft having at least one layer of human amnion and chorion tissues, and has a size and shape appropriate for covering an incision or a surgical site resulting from the surgery. Methods of preparing the construct and using it in an OB/GYN surgery are also described. The products and methods improve the performance of the OB/GYN surgery, e.g., by reducing adhesions, scar formation while also reducing inflammation and risk of post-operative infection.

### CROSS-REFERENCE TO RELATED APPLICATIONS

This application is entitled to priority pursuant to 35 U.S.C. §119(e) to U.S. Provisional Patent Application No. 61/608,397, filed Mar. 8, 2012, which is hereby incorporated by reference herein in its entirety.

### BACKGROUND OF THE INVENTION

1. Field of Invention

Embodiments of the present invention relate to methods and products for improving OB/GYN surgery. In particular, embodiments of the present invention relate to constructs comprising an allograft having at least one layer of amnion and chorion tissues for use during OB/GYN surgeries. The constructs are adapted for the ease of use for covering the surgical site or incisions resulting from the surgery.

2. Background of the Invention

Obstetrics and gynecology, or OB/GYN surgery, has been used broadly for the treatment or diagnosis of diseases affecting the female reproductive organs. The surgery involves a surgical procedure conducted on a region or area in a female reproductive organ, such as the uterus and the ovary. Complications of OB/GYN surgery include, but are not limited to, bleeding, infections or inflammations, etc.

The amnion is a thin, cellular, extraembryonic membrane that forms the inner membrane of a closed placental sac surrounding and protecting an embryo in reptiles, birds, and mammals. The sac contains the fetus and amniotic fluid or liquor amnii, in which the embryo is immersed, nourished and protected. Amnion is a tough, transparent, nerve-free, and nonvascular membrane consisting of two layers of cells: an inner, single-cell-thick layer of ectodermal epithelium and an outer covering of mesodermal, connective, and specialized smooth muscular tissue. In the later stages of pregnancy, the amnion expands to come in contact with the inner wall of the chorion creating the appearance of a thin wall of the sac extending from the margin of the placenta. The amnion and chorion are closely applied, though not fused, to one another and to the wall of the uterus. Thus, at the later stage of gestation, the fetal membranes are composed of two principal layers: the outer chorion that is in contact with maternal cells and the inner amnion that is bathed by amniotic fluid.

The amnion has multiple functions, e.g., as a covering epithelium, as an active secretary epithelium, and for intense intercellular and transcellular transport. Before or during labor, the sac breaks and the fluid drains out. Typically, the remnants of the sac membranes are observed as the white fringe lining the inner cavity of the placenta expelled after birth. The amnion can be stripped off from the placenta. The amnion has a basement membrane side and a stroma side.

The fetal membrane including amnion and chorion has been used in surgeries documented as early as 1910. See Trelford et al., 1979, Am J Obstet Gynecol, 134:833-845. Amnioplastin, an isolated and chemically processed amniotic membrane, was used for continual dural repair, peripheral nerve injuries, conjunctival graft and flexor and muscle repair. See e.g., Chao et al., 1940, The British Medical Journal, March 30. The amnion has been used for multiple medical purposes, e.g., as a graft in surgical reconstruction forming artificial vaginas or over the surgical defect of total glossectomy, as a dressing for burns, on full-thickness skin wounds or in omphalocele, and in the prevention of meningocerebral adhesions following head injury or tissue adhesion in abdominal and pelvic surgery.

In recent years, there have been renewed interests in the application of amnion in ocular surface reconstruction, for example, as an allograph for repairing corneal defects. See, for example, Tsai and Tseng, Cornea. 1994 September; 13(5):389-400; and Dua et al., Br. J. Ophthahnol 1999, 83:748-20 752. In addition, amnion and amniotic fluid have recently been used as sources of placental stem cells. See, e.g., U.S. Pat. No. 7,255,879 and WO 200073421.

Despite the clinical and published record regarding the safety and efficacy of amnion in broad surgical use, issues regarding reproducibility, safety and the precise form of amnion for each prospective indication have prevented amnion from achieving broad commercial distribution.

There is a need of improved methods and products for OB/GYN surgeries that would enhance wound healing, effectively reduce inflammation and inhibit fibroblast formation, scarring and adhesion formation. The present invention relates to such improved methods and products.

It is now discovered that using amnion in OB/GYN surgeries as described in the present invention significantly reduces inflammation and tissue adhesion, promotes uniform re-growth and epithelialization, prevents scar tissue formation, thus significantly improves performance and reduces complications of OB/GYN surgeries.

### BRIEF SUMMARY OF THE INVENTION

In one general aspect, the present invention relates to a construct for use in an OB/GYN surgery. The construct comprises an allograft comprising at least one layer of human amnion and chorion tissues, wherein the construct has a size and shape appropriate for covering an incision or a surgical site resulting from the surgery.

In another general aspect, the present invention relates to a method of preparing a construct for use in an OB/GYN surgery. The method comprises drying an allograft comprising at least one layer of human amnion and chorion tissues over a frame, preferably a rigid or semi rigid frame, of a shape appropriate for covering an incision or a surgical site resulting from the surgery.

Another general aspect of the present invention relates to an improved OB/GYN surgery. The improvement comprises covering an incision or a surgical site resulting from the surgery with an allograft comprising at least one layer of human amnion and chorion tissues, wherein the construct has a size and shape appropriate for covering the incision or the surgical site.

Yet another general aspect of the present invention relates to a kit, which comprises:

(a) a plurality of constructs for use in an OB/GYN surgery according to embodiments of the present invention; and

(b) instructions on covering the incision or the surgical site resulting from the surgery with the constructs,

wherein at least two of the constructs are different in at least one of size and shape.

Preferably, the kit further comprises an amniotic fluid and instructions on applying the amniotic fluid to cover the incision or the surgical site during the surgery.

In a preferred embodiment of the present invention, the human amnion and chorion tissues and amniotic fluid are obtained by a process comprising:
(a) obtaining informed consent from pregnant females;
(b) conducting risk assessment on the consented pregnant females to select an amnion donor;
(c) procuring after birth placenta from the amnion donor; and
(d) obtaining amnion and chorion tissues from the placenta.

According to other embodiments of the present invention, the improvement to an OB/GYN surgery further comprises applying an amniotic fluid to the incision or the surgical site to thereby cover the incision or the surgical site with the amniotic fluid, and the kit further comprises an amniotic fluid and instructions on how to use the amniotic fluid in the OB/GYN surgery.

Other aspects, features and advantages of the invention will be apparent from the following disclosure, including the detailed description of the invention and its preferred embodiments and the appended claims.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 illustrates a construct according to an embodiment of the present invention that can be used in an OB/GYN surgery, which has round corners and a general arc shape;

FIG. 2 illustrates constructs according to embodiments of the present invention that can be used in an OB/GYN surgery, which have round corners and can optionally contain an open space in the middle;

FIG. 3 illustrates a construct according to an embodiment of the present invention that can be used in an OB/GYN surgery, which has round corners; and

FIG. 4 illustrates a construct according to an embodiment of the present invention that can be used in a myomectomy, which contains multiple adjacent parabolic shapes.

### DETAILED DESCRIPTION OF THE INVENTION

Unless defined otherwise, all technical and scientific terms used herein have the same meaning as commonly understood to one of ordinary skill in the art to which this invention pertains. In this application, certain terms are used, which shall have the meanings as set forth in the specification. It must be noted that as used herein and in the appended claims, the singular forms “a,” “an,” and “the” include plural reference unless the context clearly dictates otherwise.

In one general aspect, the present invention relates to a construct for use in an OB/GYN surgery. The construct comprises an allograft comprising at least one layer of human amnion and chorion tissues, wherein the construct has a size and shape appropriate for covering an incision or a surgical site resulting from the surgery, thereby providing anti-adhesion, anti-microbial and anti-inflammatory functions to improve the surgery.

Embodiments of the present invention can be used to improve the performance of any therapeutic or diagnostic procedure that involves a region or area of a female reproductive organ, including, but not limited to, the cervix, uterus, ovaries, vagina, and fallopian tubes. Examples of such procedures include, but are not limited to, cesarean procedures, such as classical cesarean section, low cervical cesarean section, extraperitoneal cesarean section, cesarean section of other specified type, and other cesarean section of unspecified type.

The present invention can also be used in hysterectomy procedures, such as, subtotal abdominal hysterectomy, other and unspecified hysterectomy, total abdominal hysterectomy, other and unspecified total abdominal hysterectomy, vaginal hysterectomy, other and unspecified vaginal hysterectomy, radical abdominal hysterectomy, radical vaginal hysterectomy, laparoscopic supracervical hysterectomy (LSH), other and unspecified subtotal abdominal hysterectomy, laparoscopic total abdominal hysterectomy, other and unspecified total abdominal hysterectomy, laparoscopically assisted vaginal hysterectomy (LAVH), other and unspecified vaginal hysterectomy, laparoscopic radical abdominal hysterectomy, other and unspecified radical abdominal hysterectomy, laparoscopic radical vaginal hysterectomy (LRVH), other and unspecified radical vaginal hysterectomy, etc.

The present invention can also be used in the treatment of gynecological conditions such as endometriosis, ectopic pregnancy, and infertility, or a myomectomy to remove uterine fibroid tumors.

According to embodiments of the present invention, an allograft comprises at least one layer of amnion and chorion tissues. It can have a single layer of amnion or chorion, more than one layer of amnion or chorion, or a combination of one or more layers of amnion and one or more layers of chorion. When the allograft is a combination of one or more layers of amnion and one or more layers of chorion, the layers can be arranged in any order. The multiple layers in the allograft can be subject to a cross-linking treatment to make the layers closely adhere to each other in an integrated form. The allograft is preferably has a thickness 0.02 mm to 0.10 mm when it is to be applied to a surgical site of the female reproductive organ.

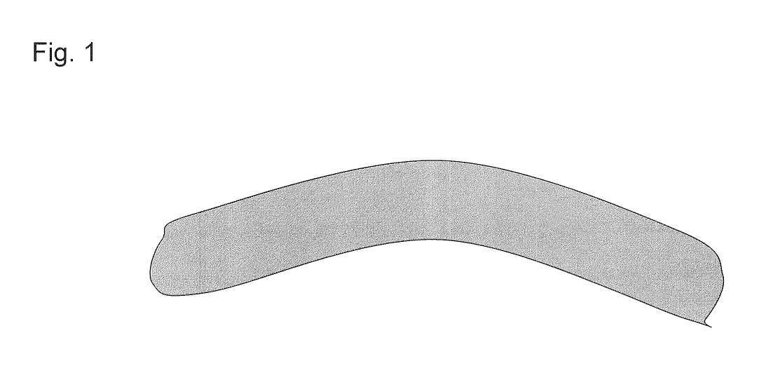

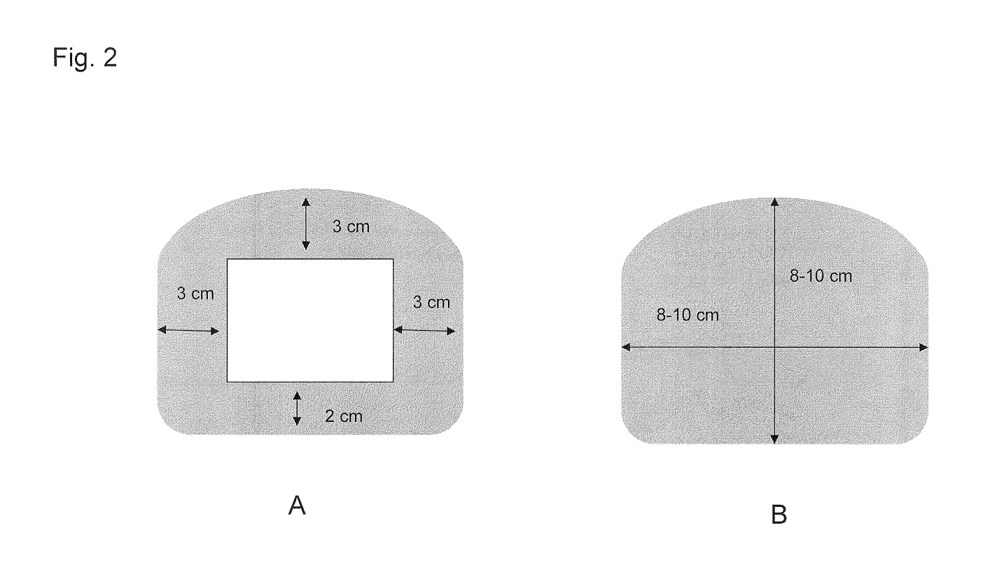

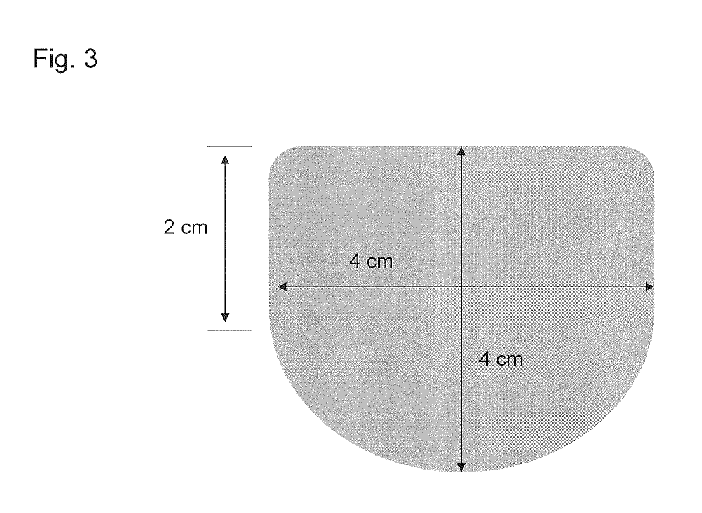

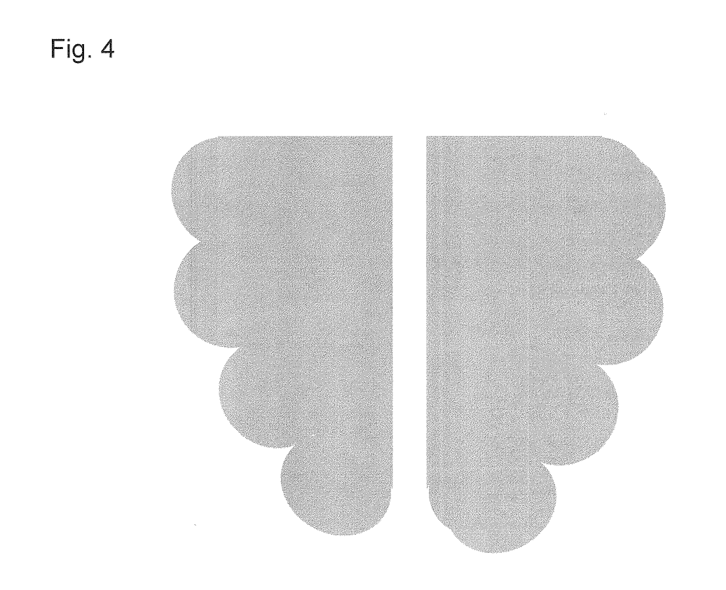

The size and shape of a construct according to an embodiment of the present invention can vary depending on the OB/GYN surgery the construct is to be used. Constructs of various sizes and shapes that can be used in embodiments of the present invention are illustrated in FIGS. 1-4. For example, as shown in FIG. 4, a construct having multiple adjacent parabolic shapes can be used in a myomectomy to remove fibroids in the uterus. The construct allows a surgeon to fold the parabolic shapes in uterus, e.g., folding them around tubes or other adjacent structures to thereby better localize the construct to a desired position. The construct can also have an arc, cylindrical or parabolic shape. The construct can also have a cutout or open space in the middle as shown in, e.g, FIG. 2A.

As readily appreciated by those skilled in the art in view of the present disclosure, depending on the size and shape of the surgery site, the construct to be used in an OB/GYN surgery may have different size and shape.

In one embodiment of the present invention, the construct for use in an OB/GYN surgery can further comprise a frame, preferably a rigid or semi rigid frame for ease of application to the surgical site. The frame can be a resorbable frame, e.g., polymer mesh frame, or a disposable or a stainless steel frame.

In one embodiment of the present invention, one or more corners of the construct or allograft are rounded or flattened to prevent the corners from catching during implantation. In view of the present disclosure, any method known to those skilled in the art can be used to make the corners of the construct or allograft round or flatten.

In one embodiment of the present invention, the allograft in the construct can carry one or more therapeutic agents, such as growth enhancing agents, morphogenic proteins, small molecule compounds, pharmaceutical agents, anti-microbial agents, anti-inflammatory agent, agents that prevent scarring, adhesions and tethering of internal tissue at or near the surgery site, analgesics, etc., to further improve the performance and reduce the complications of OB/GYN surgeries. Examples of the growth enhancing agent include, but are not limited to, growth hormone, insulin like growth factor I, keratinocyte growth factor, fibroblast growth factor, epidermal growth factor, platelet derived growth factor and transforming growth factor, and a combination of any of the foregoing.

In another general aspect, embodiments of the present invention relate to a method of preparing a construct for use in an OB/GYN surgery. The construct can be made by drying an allograft of amnion and/or chorion membranes into the required shape over a frame, such as a resorbable frame, e.g., polymer mesh frame, or a disposable or stainless steel frame. Preferably, the frame is rigid or semi rigid. The frame can be any of the shapes suitable for the surgery, e.g., arc, parabolic, cylindrical, etc.

In an embodiment of the present invention, when a disposable frame is used, the dried tissue retains the shape of the frame when removed from the frame. It could be packaged and sterilized with or without the disposable frame. The disposable frame can be removed and discarded prior to the use of the tissue. The disposable frame can be longer than the tissue for ease of handling and removal.

In another embodiment of the present invention, an implantable and resorbable frame is used. Such frame could be a mesh or a solid frame with several holes throughout. In this embodiment, the construct can be dried over the frame and packaged together with the frame. The implantable and resorbable frame can then be used in application of the tissue of the surgical site. The tissue can be hydrated on the frame prior to use of the tissue.

The allograft, such as that comprising one or more layers of human amnion and/or chorion tissues, is bonded to the frame by various methods in view of the present disclosure, such as, drying the tissue on the frame, using a resorbable adhesive, keeping the tissue wet and laying it on the frame, or freezing the tissue on the frame.

Another general aspect of the present invention relates to an improved method of performing an OB/GYN surgery. The improvement comprises covering an incision or a surgical site resulting from the surgery with an allograft comprising at least one layer of human amnion and chorion tissues, wherein the construct has a size and shape appropriate for covering the incision or the surgical site.

An improved method of performing and OB/GYN surgery according to embodiments of the present invention can result in the significant reduction of inflammation and tissue adhesion, promote uniform re-growth and epithelialization, improve healing, prevent microbial infection, prevent scar tissue formation, thus significantly improving performance and reducing complications of OB/GYN surgeries.

According to embodiments of the present invention, when a construct comprising both amnion and chorion tissues is used in an improved method of performing OB/GYN surgery, either the amnion tissue or the chorion tissue can be in direct contact with the incision or surgical site to which it is applied. In addition amnion tissue has two surfaces: (1) an outer surface in contact with chorion tissue; and (2) an inner surface in contact with amniotic fluid. Likewise, chorion tissue also has two surfaces: (1) an outer surface that is contact with maternal cells; and (2) and inner surface that is in contact with amnion tissue. According to another embodiment of the present invention, either surface (ie. inner or outer) of either tissue of the allograft (ie. amnion or chorion) can be placed in direct contact with the incision or surgical site to which it is applied.

The improvement can be applied to any OB/GYN surgical procedure in view of the present disclosure. The circumference of the allograft can be larger than the incision or surgical site it will be implanted on so that when hydrated it will fully encase the surgical site. The circumference of the allograft can also be of the same size as the incision or surgical site it will be implanted on.

In an embodiment of the present invention, a construct comprising at least one layer of amnion and chorion tissues is used to cover a skin incision resulting from an OB/GYN surgery. The allograft patch can be of any size or shape suitable for covering the sutures or other type of tissue injuries at the skin incision.

Preferably, a relatively thick layer of allograft is used to cover the skin incision. In one embodiment of the invention, the allograft patch has a thickness of about 2 mm to 4 mm. It can have multiple layers of amnion or a combination of multiple layers of amnion and chorion in any combination of amnion and chorion, and layered in any order.

In another embodiment of the present invention, amniotic fluid can be applied to the incision or surgical site to thereby cover the incision or surgical site with the amniotic fluid. The amniotic fluid can also be applied to cover a skin incision resulting from the surgery.

The amniotic fluid and the construct can be applied individually or in combination during the surgery. Preferably, the amniotic fluid is processed so that it has a relatively high viscosity for ease of application and for remaining in the desired area after the application. Methods known to those skilled in the art can be used to prepare amniotic fluid with a relatively high viscosity in view of the present disclosure. When amniotic fluid is applied in combination with a construct during surgery, the amniotic fluid is preferably applied prior to application of the construct.

The present invention overcomes shortcomings of the prior art by making human allograft membranes usable as surgical implants in an OB/GYN surgery.

There are several attributes which make an allograft having at least one of amnion and chorion tissues a preferred material for use in an OB/GYN surgery. Amnion has a complete lack of surface antigens, thus does not induce an immune response when implanted into a ‘foreign’ body, which is in contrast to most other allograft implants. Amnion also markedly suppresses the expression of the pro-inflammatory cytokines, IL-1α and IL-1β (Solomon et al., 2001, Br J. Ophthalmol. 85(4):444-9) and produces natural inhibitors of matrix metalloproteases (MMPs) expressed by infiltrating polymorphonuclear cells and macrophages. Hao et al., 2000, Cornea, 19(3):348-52; Kim et al., 2000, Exp Eye Res. 70(3):329-37). Amnion also down-regulates TGF-β and its receptor expression by fibroblasts leading to the ability to modulate the healing of a wound by promoting tissue reconstruction. Furthermore, amnion and chorion contain antimicrobial compounds with broad spectrum activity against bacteria, fungi, protozoa, and viruses for reduced risk of post-operative infection. All of these characteristics of amnion make it a potential allograft candidate to be used in an OB/GYN surgery.

Human allograft amnion and chorion have the ability to prevent scarring, reduce inflammation, inhibit microbial infection and improve healing. The allografts have the ability to reduce adhesions, scar formation while also reducing inflammation and risk of post-operative infection.

Amnion, chorion and amniotic fluid used in the present invention can be prepared from birth tissue procured from a pregnant female. Informed consent is obtained from a pregnant female by following guidelines as promulgated by the American Association of Tissue Banks and consistent with guidelines provided the Food and Drug Administration: a federal agency in the Department of Health and Human Services established to regulate the release of new medical products and, finally, if required by an established review body of the participating hospitals or institutions. The pregnant female is informed that she will be subject to risk assessment to determine if she is qualified as a birth tissue donor. She will also be informed of the tests for the risk assessment. The pregnant female is further informed that, if she is selected as a birth tissue donor based on the risk assessment, her birth tissues, such as placenta and amniotic fluid, may be collected at birth, tested and processed for medical uses.

The informed consent includes consent for risk assessment and consent for donation of birth tissues.

Risk assessment is conducted on a pregnant female with informed consent to evaluate her risk factors for communicable diseases, such as human immunodeficiency virus (HIV), hepatitis B virus (HBV), hepatitis C virus (HCV), cytomegalovirus (CMV), human T-lymphotropic virus (HTLV), syphilis, etc. Medical and social histories of the pregnant female, including physical exam record, and/or risk assessment questionnaire, are reviewed. Pregnant females with high risk factors for the communicable diseases are excluded.

Consent to draw blood at time of delivery and 1 to 12 months post delivery is obtained from pregnant females with low risk factors for the communicable diseases. Screening tests on communicable diseases, such as HIV 1 and 2, HCV, HbCore, syphilis, HTLV I/II, CMV, hepatitis B and C, are conducted by conventional serological tests on the blood sample obtained at birth. The initial screening tests are preferably completed within 7 days after birth. Preferably, the screening tests are conducted again on a second blood sample collected a few months post delivery, to verify the previous screening results and to allow for detection of communicable disease acquired shortly before birth, but are shown as “negative” on the previous screening tests. The second blood sample can be collected 1-12 months, preferably 6 months, post birth.

Only pregnant females with informed consent who are tested negative for the communicable diseases are approved as birth tissue donor. In a preferred embodiment, only pregnant females with informed consent who are tested negative for the communicable diseases in both screening tests with the blood sample drawn at birth and the blood sample drawn 6 months post delivery are approved as birth tissue donor.

Sterile techniques and procedures should be used as much as practically possible in tissue handling, e.g., during tissue procurement, banking, transfer, etc., to prevent contamination of the collected tissues by exogenous pathogens.

Only birth tissues procured from the approved birth tissue donors are subject to the collection and subsequent processing. Birth tissues, such as placenta and amniotic fluid, are recovered from the delivery room and are transferred to a location in a sterile container, such as a sterile plastic bag or bottle. Preferably, the tissues are transferred in a thermally insulated device at a temperature of 4° to 28° C., for example, in an ice bucket.

According to an embodiment of the invention, shortly after its expulsion after birth, a suitable human placenta is placed in a sterile bag, which is placed in an ice bucket, and is delivered to another location. The placenta is rinsed, e.g., with sterile saline, to removed excessive blood clots. Preferably, the placenta is subject to aseptic processing, for example, by including one or more antibiotics, such as penicillin and/or streptomycin, in the rinse. The aseptically processed placenta is stored in a controlled environment, such as hypothermic conditions, to prevent or inhibit apoptosis and contamination.

The processed placenta is placed in a sterile container, such as one made of triple sterile plastic bags, packed in wet ice, and shipped to a location for subsequent processing via overnight courier. The placenta is shipped together with release documents for processing. For example, each shipment must include technical approval to process based upon a satisfactory review of the criteria for donor selection and donor approval. The shipment must also include results on screening of communicable diseases. Preferably, the shipment includes medical director review and approval of donor eligibility/suitability.

Upon receiving the shipment and a satisfactory review of the accompanying release documents, the amnion is separated from the chorion and other remaining tissues of placenta using methods known in the art in view of the present disclosure. For example, the amnion can be stripped off mechanically from the placenta immersed in an aseptic solution, e.g., by tweezers. The isolated amnion can be stored in a cryoprotective solution comprising a cryoprotective agent, such as dimethyl sulfoxide (DMSO) and glycerol, and cryopreserved by using a rapid, flash-freeze method or by controlled rate-freeze methods. Preferably, the isolated amnion is treated with one or more antibiotics, such as penicillin and/or streptomycin, prior to cryopreservation. The chorion can also be separated from the other tissues, preserved and stored for future use.

The isolated amnion is a tough, transparent, nerve-free and nonvascular sheet of membrane. It can be dried or lyophilized using various methods. For example, it can be dried over a sterile mesh, for example, by being placed on a sterile nitrocellulose filter paper and air dried for more than 50 minutes in a sterile environment. It can also be dried or lyophilized over other form of supporting material, which would facilitate the subsequent manipulation of the amnion, such as sterilizing, sizing, cataloging, and shipping of the amnion.

The present invention encompasses a kit comprising a construct for use in an OB/GYN surgery and instructions on how to use the construct in the OB/GYN surgery. Any of the constructs for use in an OB/GYN surgery according to embodiments of the present invention can be included in the kit. The construct comprises an allograft comprising at least one layer of human amnion and chorion tissues. The construct has a shape appropriate for covering an incision or a surgical site from an OB/GYN surgery. In a preferred embodiment, the kit comprises a plurality of constructs for OB/GYN surgery, and at least two of the plurality of constructs have different shapes or sizes suitable for covering different surgical sites. The allograft in the construct can further comprise one or more therapeutically active agents, such as anti-microbial agents, growth enhancing agents, anti-inflammatory agents, analgesics, etc.

According to an embodiment of the present application, the kit further comprises an amniotic fluid and instructions on how to use the amniotic fluid in the OB/GYN surgery. Preferably, the amniotic fluid is processed amniotic fluid having a relatively high viscosity.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I/We claim:

1. A construct for use in an OB/GYN surgery, the construct comprising an allograft comprising at least one layer of human amnion and chorion tissues, wherein the construct has a size and shape appropriate for covering an incision or a surgical site resulting from the OB/GYN surgery.

2. The construct of claim 1, further comprising a rigid or semi rigid frame, the frame being disposable or implantable and resorbable.

3. The construct of claim 1, having an arc shape, a cylindrical shape or a parabolic shape.

4. The construct of claim 1, having multiple adjacent parabolas and being adapted for use in a myomectomy.

5. The construct of claim 1, further comprising one or more therapeutic agents to further improve the performance and reduce the complications of the OB/GYN surgery.

6. The construct of claim 5, wherein the one or more therapeutic agents are selected from the group consisting of growth enhancing agents, morphogenic proteins, anti-microbial agents, anti-inflammatory agent, agents that prevent scarring, adhesions and tethering of internal tissue at or near the surgery site, and analgesics.

7. A method of preparing a construct for use in an OB/GYN surgery, the method comprising drying an allograft comprising at least one layer of human amnion and chorion tissues over a frame of a shape appropriate for covering an incision or a surgical site resulting from the OB/GYN surgery.

8. The method of claim 7, wherein the frame has an arc shape, a cylindrical shape or a parabolic shape.

9. The method of claim 7, wherein the human amnion and chorion tissues are obtained by a process comprising:
(a) obtaining informed consent from pregnant females;
(b) conducting risk assessment on the consented pregnant females to select an amnion donor;
(c) procuring after birth placenta from the amnion donor; and
(d) obtaining amnion and chorion tissues from the placenta.

10. A method of performing an improved OB/GYN surgery, the improvement comprising covering an incision or a surgical site resulting from the surgery with an allograft comprising at least one layer of human amnion and chorion tissues, wherein the construct has a size and shape appropriate for covering the incision or the surgical site.

11. In the method of claim 10, the construct further comprising a rigid or semi rigid frame.

12. In the method of claim 10, the improvement further comprising applying an amniotic fluid to the incision or the surgical site to thereby cover the incision or the surgical site with the amniotic fluid.

13. In the method of claim 10, the construct having an arc shape, a cylindrical shape or a parabolic shape.

14. In the method of claim 10, the OB/GYN surgery being a myomectomy and the construct having multiple adjacent parabolas.

15. In the method of claim 10, the improvement thereby resulting in reduced scarring, reduced inflammation, or reduced risk of infection at the incision or surgical site.

16. In the method of claim 10, the construct further comprising one or more therapeutic agents to further improve the performance and reduce complications of the OB/GYN surgery.

17. A kit comprising
(a) a plurality of constructs for use in an OB/GYN surgery according to claim 1; and
(b) instructions on covering the incision or the surgical site resulting from the surgery with the constructs,
wherein at least two of the constructs are different in at least one of size and shape.

18. The kit of claim 17, further comprising an amniotic fluid and instructions on applying the amniotic fluid to cover the incision or the surgical site during the surgery.

19. The kit of claim 18, wherein the amniotic fluid is processed amniotic fluid having a relatively high viscosity.
